(19) United States
(12) Patent Application Publication
(10) Pub. No.: US 2015/0140088 A1
(43) Pub. Date: May 21, 2015

(54) SPIROSTEROID SYSTEMS ACTING AS NEUROPROTECTIVE AND ANTI-INFLAMMATORY AGENTS

(71) Applicants: Laura Garcia Pupo, La Habana (CU); Yanier Nuñez Figueredo, Ciudad Habana (CU); Juan Enrique Tacoronte Morales, Ciudad Habana (CU); Yamila Verdecia Reyes, La Habana (CU); Estael Ochoa Rodriguez, Ciudad de la Habana (CU); Zaldo Castro Armando, Ciudad de La Habana (CU)

(72) Inventors: Laura Garcia Pupo, La Habana (CU); Yanier Nuñez Figueredo, Ciudad Habana (CU); Juan Enrique Tacoronte Morales, Ciudad Habana (CU); Yamila Verdecia Reyes, La Habana (CU); Estael Ochoa Rodriguez, Ciudad de la Habana (CU); Zaldo Castro Armando, Ciudad de La Habana (CU)

(21) Appl. No.: 14/368,668

(22) PCT Filed: Dec. 27, 2012
(86) PCT No.: PCT/CU2012/000008

§ 371 Date: Jun. 25, 2014

(30) Foreign Application Priority Data
Dec. 27, 2011 (CU) P/2011/0244

(51) Int. Cl. C07J 71/00; C07J 43/00; C07J 33/00; C07J 21/00

(52) U.S. Cl. CPC C07J 71/0005; C07J 21/00; C07J 43/006; C07J 33/005
USPC 424/456; 514/89; 514/95; 514/100; 514/173; 540/16; 540/17; 540/5; 424/451

(57) ABSTRACT

The present invention relates to the fields of chemistry and pharmacy and, in particular, to the production of novel molecular entities: esterane derivatives fused with spirostanes rings, acting upon the Central Nervous Systems (CNS). From diosgenin, a naturally occurring sapogenin, with some subsequent transformations thereof, spirosteroid derivatives of the I-IV general formula can be obtained, with a cyclopentaneperhydrophenantrene nucleus fused to a 25R-spirostanes nucleus. Such molecular entities have an anti-inflammatory and anti-glutamatergic actions that can be used to treat inflammatory, cerebrovascular, neurodegenerative, neuropsychiatric, and neurologic diseases.

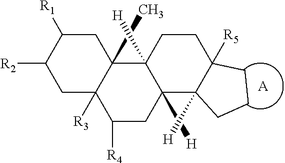

### PRIOR RELATED APPLICATIONS

This application is a 371 U.S. National Phase Patent Application which claims priority to PCT Patent Application No. PCT/CU2012/000008, filed Dec. 27, 2012 and Cuba Patent Application CU/P/2011/0244, filed Dec. 27, 2011 and incorporates the above-referenced applications in their entireties by reference thereto.

### DESCRIPTION OF THE INVENTION

This invention relates to the chemical and pharmaceutical branches, and more specifically with obtaining new molecular entities, synthetic variants of steranes fused steroids of a general formula:

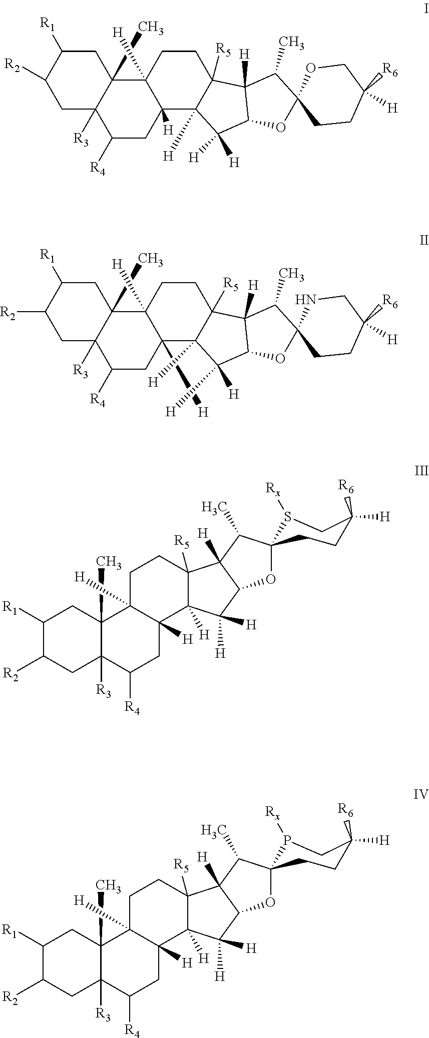

For compounds of general formula I, II, III and IV, R₁, R₂ and R₄ represents H, hydroxyl, cetal, alcoxyl, alkanyloxyl, alkenoxyl and alkoxylcarbonyloxyl groups (preferable alkyl groups having up to 8 carbon atoms such as methyl, ethyl, propyl, butyl, pentyl, hexyl, heptyl, and octyl and all chain isomers thereof).

For compounds of general formula I, II, III and IV, R₁, R₂ and R₄ also represents an amine group, preferable substituted with alkylamines, dialkylamines, alkenylamines, dialkenylamines, aminecarbonyloxyl, alkinylamines and dialkinylamines groups (preferable alkyl groups having up to 8 carbon atoms such as methyl, ethyl, propyl, butyl, pentyl, hexyl, heptyl, and octyl and all chain isomers thereof).

For compounds of general formula I, II, III and IV, R₁, R₂ and R₄ also represents amide, thiol, sulphinyl, sulphonamide and sulphonyl groups, preferable substituted with alkylaryl, alkanoyloxyaryl, alkanoyloxyaryl and alkenylaryl groups (preferable alkyl groups having up to 8 carbon atoms such as methyl, ethyl, propyl, butyl, pentyl, hexyl, heptyl, and octyl and all chain isomers thereof).

For compounds of general formula I, II, III and IV, R₁, R₂ and R₄ also represents an cyane, thiocyane, isothiocyane groups, preferable substituted with alkyl, acidalkyl, alkanyloxylalkyl, arylalkyl, heteroarylalkyl, arylalkenyl, heteroarylalkenyl, arylalkinyl, arylalkylalkinyl, alkanoyloxyarylalkylalkinyl, heteroaryloxyalkinyl, heteroaryloxyalkinyl, oxoalkinyl, or cetal groups, wherein the cyanilalkinyl substituents can be substituted in turn by heteroarylalkinyl, hydroxyalkinyl, alcoxyalkinyl, aminoalkinyl and acyloaminoalkinyl groups (preferable alkyl groups having up to 8 carbon atoms such as methyl, ethyl, propyl, butyl, pentyl, hexyl, heptyl, and octyl and all chain isomers thereof).

For compounds of general formula I, II, III and IV, R₁, R₂ and R₄ also represents a phosphoryl group, preferable substituted with alkylaryl, alkanyloxyaryl, alkenyloxyaryl and alkenylaryl groups (preferable alkyl groups having up to 8 carbon atoms such as methyl, ethyl, propyl, butyl, pentyl, hexyl, heptyl, and octyl and all chain isomers thereof).

For compounds of general formula I, II, III and IV, R₃ represents an H or hydroxyl group.

For compounds of general formula I, II, III and IV, R₅ represents H, hydroxyl, cetal, amine, thiol and cyane groups.

For compounds of general formula I, II, III and IV, R₆ represents methyl, lipid chains derived from mono or polyunsaturated fatty acids having up to 24 carbon atoms and proteins union site groups.

For compounds of general formula III and IV, Rₓ represents alkyl groups having up to 8 carbon atoms such as methyl, ethyl, propyl, butyl, pentyl, hexyl, heptyl, and octyl and all chain isomers thereof, wherein x is different from zero.

For compounds of general formula III and IV, Rₓ also represents alkylaryl, alkanyloxyaryl, alkenyloxyaryl and alkenylaryl groups, preferable alkyl groups having up to 8 carbon atoms such as methyl, ethyl, propyl, butyl, pentyl, hexyl, heptyl, and octyl and all chain isomers thereof, wherein x is different from zero.

These novel compounds can serve as a basis for therapeutic drugs to treat anxiety, ischemia, epilepsy, hypertension and other cardiovascular, cerebrovascular, neurodegenerative, neuropsychiatric, and neurological disorders, as well as other disorders related to acute and chronic inflammation.

Compounds of I, II, III and IV type are obtained by tosylation of 3β-hydroxy group and hydroxylation of 6β-one group, in presence of carbonates. Oxidative reactions can employ Jones's reactive (CrO3-H2SO4), Collins's reactive (CrO3-acetone) or pyridinium chlorocromate, as oxidant agents according to Aburatani's method; or with oxoammonium-iodine salts.

Steroids were one of the first pharmacological entities denominated privileged anti-inflammatory structures. Generally, most steroid molecules act as immunosuppressive agents, through molecular mechanisms including rapid non-genomic and genomic effects. However some steroids, such as hydrocortisone, cortisone, prednisone, prednisolone, fludrocortisone, deoxycorticosterone, methylprednisolone, triamcinolone, paramethasone, betamethasone, dexamethasone, triamcinolone acetonide, acetoxyprenolone, among others, causes some toxicological effects in several organs. Therefore long-term clinical usage of those molecules induces different side effects, such as cellular metabolic disorders, osteoporosis, hypertension and gastrointestinal diseases.

In other words, the well-known neurosteroids have sedative, anesthetic and anti-convulsive affects in animals and humans. Such biological activity are due to the modulation of neuronal excitability, through the interaction between membrane receptors and ion channels, principally GABA_{A} receptor, which acts in a bidirectional inhibitory system connected to different areas in the CNS.

Disclosed herein are new steroid compounds and compositions and their application as pharmaceuticals for the treatment of anxiety, ischemia, epilepsy, hypertension and other cardiovascular, cerebrovascular, neurodegenerative, neuropsychiatric, and neurological disorders, as well as other disorders related to acute and chronic inflammation.

After an analysis of the structure of the molecules tested and their effects on vitality of damaged PC12, as an indicator of their neuroprotective potential, the use of synthetic variants of steroids fused with spirostanes cycles for treating cerebrovascular, neurodegenerative, neuropsychiatric and neurological diseases is justified.

The novelty in this invention is obtaining a spirosteroid molecular system for potential application in the treatment of cardiovascular, cerebrovascular, neurodegenerative, neuropsychiatric and neurological diseases, as well as the possibility of obtaining these spirosteroid systems using a simple method of synthesis and steroid sapogenin-like diosgenin, hecogenin and solasodine as starting materials.

There are several patents describing steroids derivatives for treating CNS and inflammatory diseases. In such cases, however, no description is made of the fusion of these nucleuses or the inclusion of spirostanes cycles to form a new pharmacologic entity. Patents using different substituents of the spirosteroid nucleus, having no relation with the subject matter of our invention are listed below:

### BACKGROUND OF THE PRIOR ART

Patents U.S. Pat. No. 6,909,007, U.S. Ser. No. 186,708 and U.S. Pat. No. 5,116,829 describe the process of obtaining steroid molecules as potential anti-inflammatory entities less toxic than conventional pharmaceuticals, but use hydrogen-type substituents, alkyl and alkenyl chains, and aromatic rings of the phenyl, carboxyphenyl, acetoxyphenly, methoxycarboxyphenyl and dimethylcarboxyphenyl type. U.S. Pat. No. 5,599,807 describes the process of obtaining steroid molecules with an ester group between carboxyl group of kinoloncarboxylic acid and alcoholic hydroxyl group in position 21, with anti-bacterial and anti-inflammatory activities without long-term immunosuppression.

Patent US20030092692 describes chemical production and application of known and novels 7α-hydroxy steroid molecules on in vitro cytoprotection of neuronal cells and for treatment of acute diseases affecting CNS. However, described substituents only includes H, hydroxyl, sterified hydroxyl, alkyl, amine, alkylamine and dialkylamine groups. The patents US200330186953, US20090227551 and US201001304559 also describes the process of obtaining, and application as treatment of neurological diseases, of 3-hydroxy-7β-hydroxy and some cetonide derivatives, but their described substituents omit spirostanes cycles.

Patent US20040072806 describes only the methods, combinations and applications for treatment and prevention of neurological diseases, of natural 22R-hydroxy steroid derivatives substituted with spirost-5-en-3-ol groups.

The spirosteroid synthetic variants, the subject matter of our invention, showed some kind of action upon neuronal cells. However, the degree of the action depends on the nature of the R substituent at the 3 and 6-positions and the nature of R₂ and R₄ substituents.

General experimental conditions: Fusion temperatures were determined with an Electrothermal 9100 capillary hot plate equipment. IR spectrums were registered by a Philips Analytical PU 9600 FTIR spectrophotometer in KBr tablets. The NMR 1H and NMR 13C were recorded by a Brucker ACF-250 spectrometer operating at 250.13 MHz and 62.50 MHz, respectively. All those records were determined at 26° C., with CDCl3 as solvent and TMS as internal reference. Spectral assignments in NMR were developed employing the correlating spectrum (HHCOSY y HCCOSY) for some compounds, edition DEPT technique, and comparing with spectral data of compounds reported from literature.

### EXAMPLES OF PROCEDURE

### Example 1

### Synthesis of 6β-hydroxy 6-one poly-hydroxy Derivatives

Those compounds with 3α,5-cyclo-6-one structure, were obtained through Aburatani's method, which employs the 5-en-3β-ol system as starting material and consists in three principals stages. These experimental stages are performed in a continuous way and are the following:
Tosylation of diosgenin/hecogenin.
Mesylation with MSCl, triethylamine and butanone.
Isomerization with NaHCO₃ aqueous solution to form the i-steroid.
Oxidation of C-6 hydroxyl group with Jones's reactive, to obtain the 3α,5-cyclo-6-one derivative.
Isomerization of i-systems to Δ² spirostanic systems in presence of Li₂CO₂/CaCO₃/DMFA/HMFTA.
Dihydroxylation in catalytic conditions (CHCl₃/NaOH/cetyltrimethylammonium bromide), and catalysis by solid bentonite acids and pyritic ashes.
Catalytic esterification with niobyl-vanadyl acetate/acetic anhydride.
Epoxide opening with bentonite-pyritic ashes and a reflux in water.

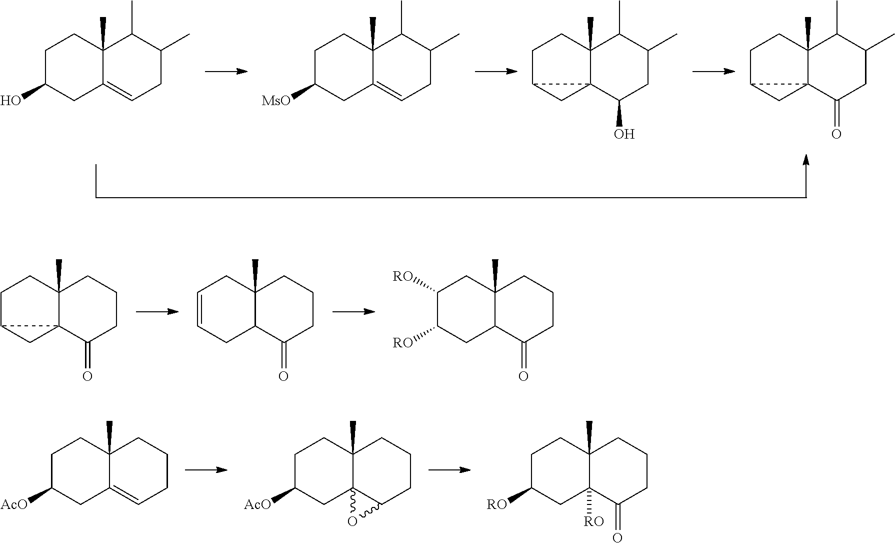

### Example 2

### Chemical and Structural Characterization of 6R-hydroxy 6-one poly-hydroxy Derivatives

The spectroscopic reported data corroborates the proposed structures. In FTIR spectrums are observed the typical bands in 1350 to 850 cm⁻¹ zones, due to vibrations of spiroketal systems, associated to narrowing in C—C and C—O bonds. These evidences demonstrate there were not structural degradation changes in E and F rings. Moreover, the band in 880-910 cm⁻¹ is about twice more intense than the band in 925-910 cm⁻¹, therefore all synthesized compounds belongs to 25R series. The frequency zone 1713-1735 cm⁻¹, corresponding to acetoxy group CH₃—CO—O—, is typical for synthesized acetoxy derivatives. The band in 1360 and 1170-1175 cm⁻¹, corresponding to asymmetric and symmetric vibrations of SO₂ group (v SO₂ s and as.), are characteristic for the tosyl derivative. To this grouping corresponds the band in 1588 cm⁻¹ assignable to an aromatic v C═C.

The NMR-¹H and ¹³C spectroscopy were informative in order to elucidate the structure of synthesized compounds. Among common significant signals are those of protons from methyl groups (CH₃) 18, 19, 21 and 27, and those of protons H₂-26 and H-16α. The H₂-26 signal is complex and corresponds to both chemical shifts H-26α and H-26β, which practically does not vary in the series of studied compound. The axial H-26β is a triplet with δ=3.32 ppm; and its multiplicity results from a germinal quasi degenerate and vecinal axial-axial (J²≅J³=10.6 Hz) double coupling. The H-26β signal is a double doublet with δ=3.5 ppm; and its multiplicity results from a germinal coupling (J²=10.6 Hz) and vecinal equatorial-axial (J³ₑₐ=2.6 Hz). With a chemical shift of γ=4.4 ppm, the appearance of a double doublet is typical for H-16α, due to its coupling with protons 15β (equatorial), 15α and 17β (quasi-equatorials).

The signals of olefinic protons H-2 and H-3 with δ=5.5 and 5.7 ppm, respectively, are characteristics for Δ²-6-oxo steroidal compounds. The signals of 7β and 5α protons appear superpose in 2.3-2.4 ppm. In NMR-¹³C C-2 and C-3 signals are unshielded to δ=124.4 and 124.5 ppm, respectively, while C-4 shields to 21.7 ppm.

TABLE 1
Chemical shifts (δ, ppm) of carbon atoms from A and B rings,
and C-19 of (25R)-3α,5-cyclo-spirostan-6β-ol (G1) and
(25R)-2,3-dihydroxy-spirostan-6β-ona (G2).

Compounds

| Carbons # | G1 | G2 |
|---|---|---|
| 1 | 33.2 | 40.15 |
| 2 | 24.9 | 68.23 |
| 3 | — | 68.31 |
| 4 | 11.8 | 26.34 |
| 5 | 38.8 | 50.73 |
| 6 | 73.5 | 211.79 |
| 7 | 37.4 | 46.81 |
| 8 | 29.5 | 37.14 |
| 9 | 24.2 | 53.72 |
| 10 | 43.0 | 42.51 |
| 19 | 20.2 | 13.63 |

### Example 2

### Effects of the Synthetic Spirosteroids Series on Cellular Vitality of PC12 Exposed or Not to Glutamatergic Damage

PC12 constitute a neuron-like cell line widely employed in several models of neurological diseases. The patophysiological mechanisms which can be simulated in those in vitro models, allows the use of PC12 cells in evaluation of therapeutic candidates.

The synthetic spirosteroids series (10 μM of each molecule) was evaluated on PC12 cells exposed to L-glutamate (20 mM) for 24 h. Previously, those cells were routinely cultivated in RPMI 1640 medium, supplemented with equine serum (10%) and fetal bovine serum (5%) in a 37° C. and 5% CO₂ atmosphere. In the beginning of the experiment, cells were seeded in 96-well microplates at a cellular density of 150,000 cells/mL. After 24 h of exposition to glutamatergic damage and spirosteroids treatment, cellular vitality was determined with 3-(4,5-dimethylthiazol-2-yl)-2,5-diphenyltetrazolium bromide (MTT), which is metabolized by mitochondrial deshydrogenases in living cells. Our results (Table 2) demonstrate an anti-glutamatergic and neuroprotector effect of several spirosteroids molecules (S) related to some structural features. Specifically, between the most active molecules was 2α,3α-dyhydroxy-(25R)-spirostan-6-one and 3,6-dione-(25R)-spirostan. All molecules of the synthetic spirosteroids series were non cytotoxic at the concentration employed for the in vitro anti-glutamatergic assay (data no shown).

TABLE 2
Anti-glutamatergic effect of the synthetic spirosteroids series.

| Experimental | | | | | | |
|---|---|---|---|---|---|---|
| Groups | Absorbance (540 nm) | | | | | |
| Untreated | 0.557 | 0.471 | 0.519 | 0.425 | 0.649 | 0.716 |
| L-glutamate | 0.342 | 0.341 | 0.347 | 0.534 | 0.271 | 0.385 |
| S1 | 0.619 | 0.41 | 0.396 | 0.473 | 0.527 | 0.557 |
| S2 | 0.562 | 0.372 | 0.343 | 0.42 | 0.452 | 0.536 |
| S3 | 0.538 | 0.543 | 0.357 | 0.524 | 0.505 | 0.466 |
| S4 | 0.653 | 0.605 | 0.489 | 0.607 | 0.484 | 0.643 |
| S5 | 0.646 | 0.632 | 0.378 | 0.557 | 0.543 | 0.552 |
| S6 | 0.58 | 0.592 | 0.545 | 0.562 | 0.435 | 0.589 |
| S7 | 0.516 | 0.558 | 0.437 | 0.453 | 0.391 | 0.636 |
| S8 | 0.467 | 0.28 | 0.311 | 0.547 | 0.448 | 0.482 |
| S9 | 0.412 | 0.311 | 0.27 | 0.499 | 0.501 | 0.545 |
| S10 | 0.301 | 0.257 | 0.262 | 0.499 | 0.513 | 0.41 |
| S11 | 0.364 | 0.481 | 0.392 | 0.511 | 0.47 | 0.445 |
| S12 | 0.384 | 0.423 | 0.58 | 0.501 | 0.625 | 0.486 |
| S13 | 0.571 | 0.403 | 0.593 | 0.469 | 0.626 | 0.382 |
| S14 | 0.415 | 0.381 | 0.4 | 0.586 | 0.657 | 0.659 |
| S15 | 0.423 | 0.417 | 0.579 | 0.567 | 0.612 | 0.552 |
| S16 | 0.348 | 0.394 | 0.438 | 0.53 | 0.567 | 0.563 |
| S17 | 0.345 | 0.468 | 0.329 | 0.438 | 0.545 | 0.318 |
| S18 | 0.284 | 0.346 | 0.346 | 0.44 | 0.411 | 0.464 |

1. A spirosteroidal system comprising a composition having neuroactive and anti-inflammatory effects, derived from diosgenin, hecogenin and solasodine, and having spirostanic rings fused to its structures, to be used as a drug in medicine, said composition having one of the general formula: 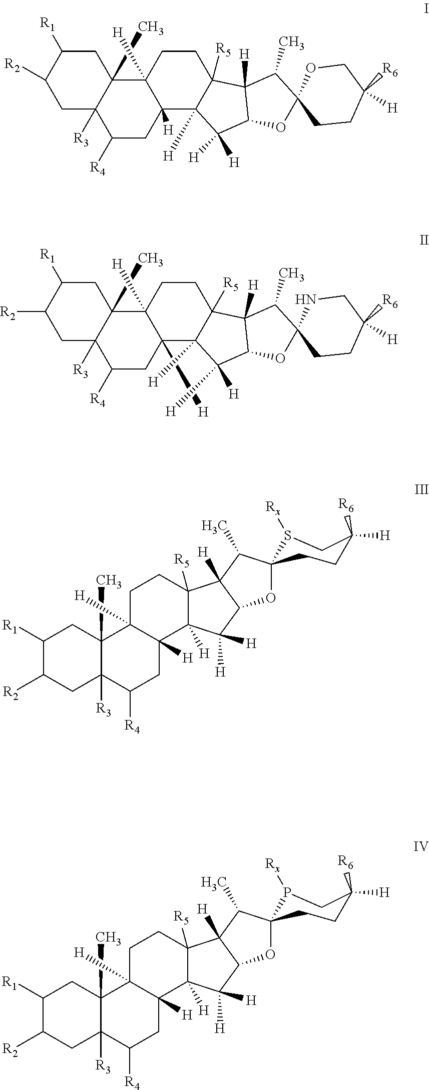
in all of its naturals conformations and configurations, wherein for compounds of the general formula I, II, III, and IV, R₁, R₂ and R₄ represents H, hydroxyl, cetal, alcoxyl, alkanyloxyl, alkenoxyl and alkoxylcarbonyloxyl groups (preferable alkyl groups having up to 8 carbon atoms such as methyl, ethyl, propyl, butyl, pentyl, hexyl, heptyl, and octyl and all chain isomers thereof);
for compounds of general formula I, II, III and IV, R₁, R₂ and R₄ also represents an amine group, preferable substituted with alkylamines, dialkylamines, alkenylamines, dialkenylamines, aminecarbonyloxyl, alkinylamines and dialkinylamines groups (preferable alkyl groups having up to 8 carbon atoms such as methyl, ethyl, propyl, butyl, pentyl, hexyl, heptyl, and octyl and all chain isomers thereof);
for compounds of general formula I, II, III and IV, R₁, R₂ and R₄ also represents amide, thiol, sulphinyl, sulphonamide and sulphonyl groups, preferable substituted with alkylaryl, alkanoyloxyaryl, alkenoyloxyaryl and alkenylaryl groups (preferable alkyl groups having up to 8 carbon atoms such as methyl, ethyl, propyl, butyl, pentyl, hexyl, heptyl, and octyl and all chain isomers thereof);
for compounds of general formula I, II, III and IV, R₁, R₂ and R₄ also represents an cyane, thiocyane, isothiocyane groups, preferable substituted with alkyl, acidalkyl, alkanyloxylalkyl, arylalkyl, heteroarylalkyl, arylalkenyl, heteroarylalkenyl, arylalkinyl, arylalkylalkinyl, alkanoyloxyarylalkylalkinyl, heteroaryloxyalkinyl, heteroaryloxyalkinyl, oxoalkinyl, or cetal groups, wherein the cyanilalkinyl substituents can be substituted in turn by heteroarylalkinyl, hydroxyalkinyl, alcoxyalkinyl, aminoalkinyl and acyloaminoalkinyl groups (preferable alkyl groups having up to 8 carbon atoms such as methyl, ethyl, propyl, butyl, pentyl, hexyl, heptyl, and octyl and all chain isomers thereon;
for compounds of general formula I, II, III and IV, R₁, R₂ and R₄ also represents a phosphoryl group, preferable substituted with alkylaryl, alkanyloxyaryl, alkenyloxyaryl and alkenylaryl groups (preferable alkyl groups having up to 8 carbon atoms such as methyl, ethyl, propyl, butyl, pentyl, hexyl, heptyl, and octyl and all chain isomers thereof);
for compounds of general formula I, II, III and IV, R₃ represents an H or hydroxyl group;
for compounds of general formula I, II, III and IV, R₅ represents H, hydroxyl, cetal, amine, thiol and cyane groups;
for compounds of general formula I, II, III and IV, R₆ represents methyl, lipid chains derived from mono or polyunsaturated fatty acids having up to 24 carbon atoms and proteins union site groups;
for compounds of general formula III and IV, Rₓ represents alkyl groups having up to 8 carbon atoms such as methyl, ethyl, propyl, butyl, pentyl, hexyl, heptyl, and octyl and all chain isomers thereof, wherein x is different from zero and;
for compounds of general formula III and IV, Rₓ also represents alkylaryl, alkenyloxyaryl, alkenyloxyaryl and alkenylaryl groups, preferable alkyl groups having up to 8 carbon atoms such as methyl, ethyl, propyl, butyl, pentyl, hexyl, heptyl, and octyl and all chain isomers thereof, wherein x is different from zero.

2. The spirosteroidal system of claim 1, wherein derivatives of the composition are obtained by synthetic methods.

3. The spirosteroidal system of claim 1, wherein synthetic derivatives of the composition are obtained by acetoxylation-tosylation-mesylation catalytic processes, catalytic epoxidation, catalytic opening of epoxides, double bond generation in the A ring of spirosteroidal system, and subsequent dihydroxylation in phase transfer mediated catalysis conditions.

4. A pharmaceutical composition comprising the composition of claim 1, in liquid form for oral administration, characterized as contains the active substance in 1-83%, sodium carboxymethylcellulose (0.7%) or hydroxypropylmethylcellulose (0.5%), sodium saccharine (0.4%), propylenglycol (5%), 70% sorbitol (10%), a flavor (0.1%) and water as dissolvent.

5. A pharmaceutical composition comprising the composition of claim 1, in capsules form for oral administration, characterized as contains the active substance in 1-40%, colloidal silicon dioxide (1%), microcrystalline cellulose (58%), magnesium stereate (0.8%), enclosed in hard gelatin capsules or hydroxypropylmethylcellulose.

6. The pharmaceutical composition comprising the composition of claim 1, in tablets form or granulates for oral administration, characterized as contains the active substance in 1-70%, colloidal silicon dioxide (1%), microcrystalline cellulose (20%), lactose (10%) for direct compression and magnesium stereate (0.8%).

7. The pharmaceutical composition comprising the composition of claims 1, in liquid form for parenteral administration, characterized as contains the active substance in 0.1-99%, sodium chloride (0.6%), sodium monobasic phosphate and potassium dibasic phosphate as pH regulators and water for injection.

8. The pharmaceutical composition comprising the composition of claim 1, in liquid form for nasal administration, characterized as contains the active substance in 0.1-97%, sodium chloride (0.6%), dextran 70 (1%), Carbopol 974 (0.5%), sodium monobasic phosphate and potassium dibasic phosphate as pH regulators and water for injection.

9. The pharmaceutical composition comprising the composition of claim 1, in sustained-release tablets form for oral administration, characterized as contains the active substance in Eudragit S 100 microencapsulated form, in 1-70%, colloidal silicon dioxide (1%), microcrystalline cellulose (20%), lactose (10%) for direct compression and magnesium stereate (0.8%).

10. The pharmaceutical composition comprising the composition of claim 1, in sustained-release liquid form for parenteral administration, characterized as contains the active substance in polylactic coglycolic acid microencapsulated form in 0.1-99%, sodium chloride (0.6%), sodium monobasic phosphate and potassium dibasic phosphate as pH regulators and water for injection.

11. A pharmaceutical composition providing neuroprotection and anti-inflammatory effects, said pharmaceutical composition comprises a molecular entity derived from diosgenin, hecogenin and solasodine, and further comprises a plurality of spirotanic rings fused in the molecular structure of the molecular entity.

12. The pharmaceutical composition of claim 11, said molecular structure further comprises a plurality of spirosteroidal bonds.
